(19) United States
(12) Patent Application Publication
(10) Pub. No.: US 2023/0110363 A1
(43) Pub. Date: Apr. 13, 2023

(54) Intraoral Device

(71) Applicant: Perrydigma Research Ltd., Tel Aviv (IL)

(72) Inventors: Shay Joseph Mamo, Ramat HaSharon (IL); Ran S. Sopher, Ramat Gan (IL); Michal Stern-Perry, Ramat HaSharon (IL)

(73) Assignee: Perrydigma Research Ltd., Tel Aviv (IL)

(21) Appl. No.: 18/078,319

(22) Filed: Dec. 9, 2022

Related U.S. Application Data
(63) Continuation-in-part of application No. PCT/IL2021/050689, filed on Jun. 9, 2021.

(30) Foreign Application Priority Data
Jun. 10, 2020 (IL) 275280

(51) Int. Cl. A61C 19/06

(52) U.S. Cl. CPC A61C 19/063

(57) ABSTRACT

An intraoral device, comprising an antiviral and/or antibacterial and/or antifungal ion releasing agent material(s) which, when said device is exposed to the oral cavity and to any gases, liquids and solids passing through the oral cavity, and is capable of inactivating viruses and/or bacteria and/or fungi penetrating into the oral cavity.

This United States Patent Application is a continuation-in-part of International Patent Cooperation Treaty Patent Application No. PCT/IL2021/050689, filed Jun. 9, 2021, which claims the benefit of Israeli Patent Application No. 275280, filed Jun. 10, 2020, each hereby incorporated by reference herein.

### I. FIELD OF THE INVENTION

The present invention relates to the field of intraoral devices. More particularly, the invention relates to an intraoral device having antiviral and/or antibacterial and/or antifungal properties.

### II. BACKGROUND OF THE INVENTION

Due to the efficient and sustainable spread of the coronavirus caused by the transmission of small droplets exhaled by infected people and subsequently inhaled into the lungs of others, the U.S. Centers for Disease Control and Prevention (CDC) has recently recommended the widescale wearing of a mask to block the transmission of coronavirus in droplets ejected during breathing.

Personal protection equipment (PPE) in general is a crucial element to personal safety against any hazardous pathogens that are airborne, waterborne, foodborne or are contracted via touch. Pathogens bacterial, viral, fungal, microbial or other can be transmitted from person to person in many different ways including, but not limited to: through droplets in the air, as a result of exhaling/inhaling, breathing, spitting, sneezing, coughing, direct touch. Pathogens can also be transmitted through water and food and due to poor hygiene.

Herein the term “microbe” refers to any type of the following: bacterium, virus, fungus, ‘germ’ or otherwise. An expert in the art will understand that, for the purposes of the present invention, these terms can be used interchangeably when referring to any contracted pathogen and/or germ that could potentially result in a disease.

Although masks covering the mouth and nostrils are beneficial in providing protection against viruses, bacteria such as the coronavirus, users are confronted with several difficulties while wearing a mask, including a difficulty in breathing, fogged eyewear, a degradation of the auditory signal and a limitation in determining the facial expressions of others during a conversation, and the psychological distancing between people and particularly between healthcare workers and patients. Mask usage is often punctuated with intermittent removal of the mask. Many mask users report the difficulty of continuous use which leads to those masks being worn sub-optimally, e.g., not fully covering the mouth and/or nostrils, which leaves the user susceptible to becoming infected. Moreover, as some infections are carried in food, air, saliva, water and hands, for example (not droplet-transmitted), wearing a mask provides no protection in such cases (e.g., when someone eats).

It is an object of the present invention to provide a passive means to protect the user (and others around him/her) by using a continuously worn device that can be removed without causing inconvenience.

It is another object of the present invention to provide reliable means other than a mask to block the transmission of viruses and/or bacteria such as the coronavirus.

It is an additional object of the present invention to provide means other than a mask to minimize the transmission of viruses and/or bacteria such as the coronavirus that does not significantly interfere with the performance of normal everyday activities. Furthermore, the device can work continuously but yet is removable. Due to the inconspicuous nature of the device it can be worn seamlessly and is considerably less likely to be removed (or used improperly) compared to common masks which are often used suboptimally. In this sense, the device serves as an effective counterpart to regular mask usage.

Other objects and advantages of the invention will become apparent as the description proceeds.

### III. SUMMARY OF THE INVENTION

The present invention provides an intraoral device, comprising antiviral and/or antibacterial and/or antifungal agent material(s) which, when said device is engaged with a dental structure, are exposed to the oral cavity in which the dental structure is located and to gases, liquids and solids passing through the oral cavity, and are capable of inactivating viruses and/or bacteria and/or fungi carried by gases, liquids and solids upon contact with the viruses and/or bacteria and/or fungi that penetrate into the oral cavity. Accordingly, a major advantage of the device is the ability to prevent the penetration of viruses (or other forms of submicroscopic infectious agent) into the user's body through the oral cavity (e.g., before approaching the lungs or other internal organs).

According to an embodiment of the invention, the device is not intended to replace masks but, rather, is complementary to mask usage. While masks are generally intended to prevent disease transmission, the device is intended to protect the user and others that are in close proximity.

According to an embodiment of the invention, the device is a solid dental clip comprises the antiviral and/or antibacterial agent material(s) that are removable and engages the dental structure.

According to an embodiment of the invention, the solid clip is a looped dental clip.

According to an embodiment of the invention, the clip is configured as a single continuous loop whose peripheral element completely encircles the dental structure.

According to an embodiment of the invention, the peripheral element completely encircles a unit of one or more teeth.

According to an embodiment of the invention, the peripheral element completely encircles a unit of one or more teeth of a set of dentures.

According to an embodiment of the invention, the peripheral element completely encircles a unit of one or more teeth of a dental bridge.

According to an embodiment of the invention, the peripheral element comprises two opposed interproximal elements, two opposed buccolingual elements, and a curved portion continuously extending between corresponding interproximal and buccolingual elements.

According to an embodiment of the invention, the device is an insert made with the antiviral and/or antibacterial agent material(s) and embedded in a base of a set of dentures that extends throughout the dental arch. According to an embodiment of the invention, the insert extends interproximally. According to an embodiment of the invention, the insert extends interproximally throughout the dental arch.

According to an embodiment of the invention, the antiviral and/or antibacterial agent material(s) are in the form of metal foils covering the shape of the tooth.

According to an embodiment of the invention, the antiviral and/or antibacterial agent material(s) are restorative material(s) which are capable of being applied to a tooth structure from which a carious lesion has been removed.

According to another embodiment of the invention the device can be installed on full or partial dentures or any other pre-existing intraoral devices. Such intraoral devices include, but are not limited to: implants, crowns, posts, bridges, retainers, braces, fillings, restorations, sealants and orthodontic devices.

According to another embodiment of the invention the antiviral/antibacterial agent can be incorporated into existing materials used to manufacture dental devices and/or other intraoral dental materials such as filling materials, alloys and amalgams that are used in dental procedures.

According to an embodiment of the invention, the device is a chewing gum made with the antiviral and/or antibacterial agent material(s).

According to an embodiment of the invention, the device is a dental sealant comprising the antiviral and/or antibacterial and/or antifungal agent material(s).

According to an embodiment of the invention, the device is adapted to be installed on full or partial dentures or any other pre-existing intraoral device, or on at least one artificial or non-artificial tooth.

According to an embodiment of the invention, the device further comprises at least one cartridge that comprises ion releasing material.

According to an embodiment of the invention, the at least one cartridge is interchangeable.

According to an embodiment of the invention, the at least one cartridge comprises combination of two or more different types of ion releasing materials.

In another aspect, the device is adapted to mechanically receive (e.g., by insertion, magnetically) at least one interchangeable cartridge or plate that comprises ion releasing material.

The intraoral device may also be provided in a malleable gum-like form detachably adhered by pressing onto a user's teeth or soft tissue lining of the mouth.

### IV. BRIEF DESCRIPTION OF THE DRAWINGS

In the drawings:

FIG. 1A illustrates an intraoral device in the form of an antiviral and/or antibacterial clip for a single tooth placed on a single tooth, according to embodiments of the present invention;

FIG. 1B illustrates an intraoral device in the form of an antiviral and/or antibacterial clip for multiple teeth placed on multiple teeth, according to an embodiment of the present invention;

FIG. 2 illustrates an enlarged view of the antiviral and/or antibacterial clip of FIG. 1B in accordance with transparent teeth view, according to an embodiment of the present invention;

FIG. 3 illustrates the intraoral device in form of a denture with an antiviral and/or antibacterial insert, according to an embodiment of the present invention;

FIG. 4A illustrates the intraoral device in form of a mouth guard with metallic strips, according to an embodiment of the invention;

FIG. 4B illustrates the mouth guard of FIG. 4A placed on the upper teeth, according to an embodiment of the invention;

FIG. 5 illustrates the intraoral device with an antiviral and/or antibacterial ion-releasing agent material(s) in the form of a metallic foil that covers the back teeth, according to an embodiment of the invention;

FIG. 6 illustrates an antiviral and/or antibacterial ion-releasing agent in the form of a filling material;

FIG. 7 schematically illustrates the intraoral device made of antiviral and/or antibacterial ion-releasing material and placed around a tooth, according to an embodiment of the invention;

FIG. 8 schematically illustrates the intraoral device provided with an interchangeable antiviral and/or antibacterial ion-releasing cartridge and placed around a tooth, according to an embodiment of the invention;

FIGS. 9A-9C schematically illustrates the intraoral device provided with a magnetic interchangeable antiviral and/or antibacterial ion-releasing plate, according to an embodiment of the invention; and

FIG. 10 schematically illustrates the intraoral device in a malleable gum-like alternate form, according to an embodiment of the invention.

### V. DETAILED DESCRIPTION OF THE INVENTION

It has now been discovered that transitional metals of group 11 and 12 of the periodic chart such as copper, zinc, and silver release ions which are effective in fighting viruses. Specifically copper ions seem to be most effective against viruses such as COVID-19.

According to some embodiments, the present invention relates to an intraoral device which is adapted to release virus-fighting ions in the oral cavity. When a person contracts a virus and/or bacterium the intraoral device, which contains one or more of the abovementioned metals, can cause the virus and/or bacteria to become inactivated. Protecting the user from pathogens at the most common points of entry to the body, namely the mouth and nostrils, provides the best line of defense in guarding against diseases.

According an embodiment of the invention, the proposed intraoral device is adapted to be detachably engaged with the teeth and/or dental structures for prolonged periods of time to ensure ongoing antiviral ions presence within the oral cavity and to avoid the risk of infection if the user contracts the viruses and/or bacteria at a time when the intraoral device is removed from the oral cavity. According to another embodiment of the invention, the proposed device is periodically replaced with a new one, or alternatively, being refreshed (e.g., it may be tarnished and therefore may release reduced quantities of antiviral ions).

The proposed device may either be intrinsically constructed with one or more of the antiviral ions releasing metals, or be adapted to exchangeably accommodate disposable insert elements (e.g., a cartridge) which contain a predetermined quantity thereof, as will be illustrated herein.

The mechanism whereby copper and silver (including many other metals and alloys) provides antimicrobial resistance is currently an active area of research. Nonetheless, the antimicrobial nature of copper and silver has been shown in numerous studies.

Various types of antiviral ion releasing metals are suitable for the invention, including solid-state copper (Cu), silver (Ag) copper(I) oxide (Cu₂O), copper(II) oxide (CuO), silver nitrate (AgNO₃), silver oxide (Ag₂O), copper(II) chloride (CuCl₂), copper(I) chloride (CuCl), Zinc-Oxide, silver nitrate (AgNO₃) and silver sulfide (Ag₂S). According to some embodiments, the aforementioned antiviral and/or antibacterial agent material(s) may be incorporated into a polymer. Also, any of the above can be incorporated into a nanoparticle coating, for example, containing particles of a diameter of 50-75 nm; this can be produced in polyvinyl alcohol (PVA) and its derivatives, for example.

All of the above materials serve to inactivate hazardous microbes. For example, Solanki G. High Copper Amalgam Alloys in Dentistry. Int J Pharmacol Res. 2012; 2(1):66-68 discloses that the high copper have become material of choice as compared to low copper alloys nowadays because of their improved mechanical properties, corrosion resistance, better marginal integrity and improved performance in clinical trial. High copper had much more strength, corrosion resistance, durability and resistance to tarnish as compared to low copper amalgams.

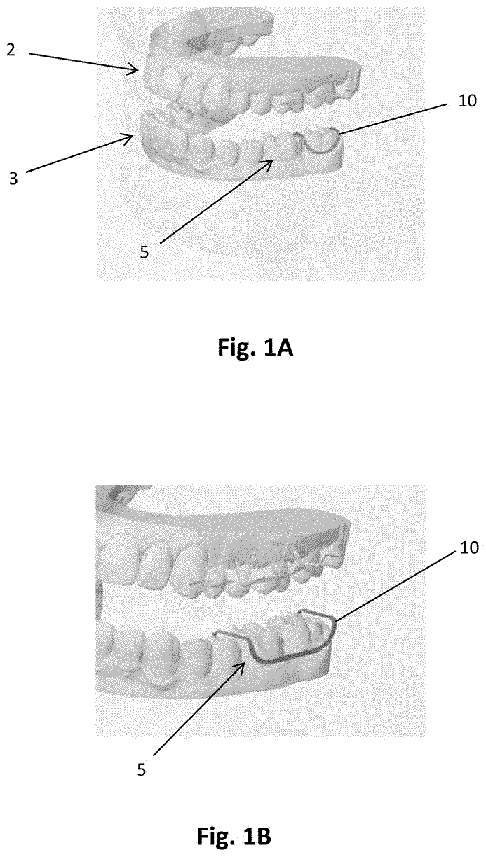

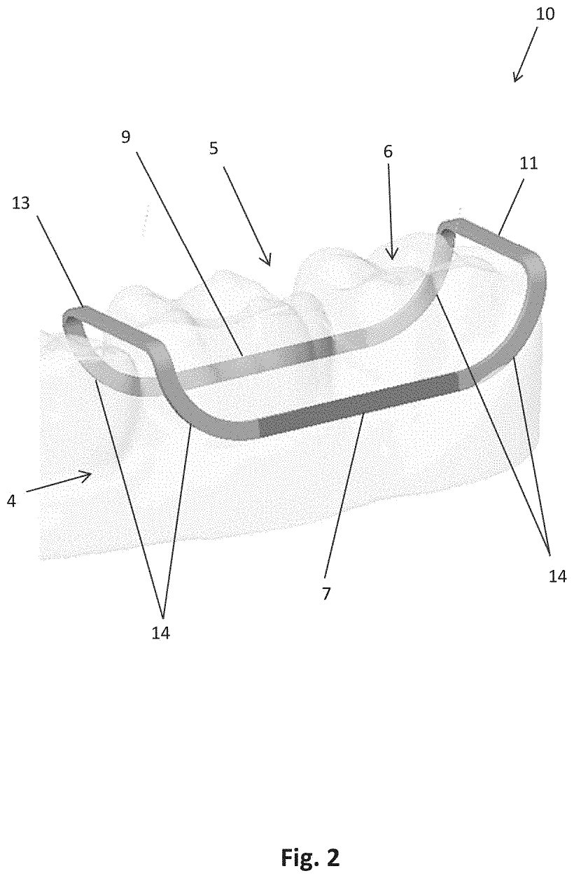

FIGS. 1A and 1B illustrate an intraoral device in form of a denture attached clip, generally indicated by numeral 10, which is made with a material that comprises an antiviral agent material(s), whether a mixture (i.e., different sections of clip 10 made from different materials as shown in FIG. 2, or clip 10 or sections thereof made with a compound alloy of two or more materials) or a single-constituent product. Clip 10 is configured as a continuous looped strip which is adapted to be secured to a dental structure 5, for example a single tooth (FIG. 1A) or several teeth (FIG. 1B), whether maxillary teeth 2 or mandibular teeth 3. A plurality of clips may be engaged with corresponding dental structures at the same time.

Clip 10 may be customized to fit the dimensions of a specific user's dental structure. Alternatively, clip 10 may be a generic product that is suitable for many different users.

Clip 10 (as best seen in FIG. 2) has two spaced interproximal elements, facial element 7 and lingual element 9, and two buccolingual elements, posterior element 11 and anterior element 13. A curved portion 14, generally concave when viewed from a coronal direction, extends continuously from an interproximal element to an adjacent buccolingual element positioned coronally with respect to the interproximal element, for example on an occlusal surface such as a mesiodistal surface thereof.

By virtue of the stiffness of the antiviral and/or antibacterial agent material(s) and the spacing between opposed elements, clip 10 is imparted with sufficient flexibility to facilitate securement to dental structure 5. If so desired, the interproximal elements 7 and 9 and the buccolingual elements 11 and 13 may be made of a rigid material, and the curved portions 14 may be made of a flexible material. Alternatively, the entire clip 10 may be made of a flexible material.

The looped strip configuration covers only a small fraction of dental structure 5, and therefore does not interfere with the functionality of dental structure 5. In the illustrated manner that clip 10 is engaged with dental structure 5, for example, by which interproximal elements 7 and 9 are positioned at the gingival margin 4 and the occlusal surfaces 6 are completely uncovered by buccolingual elements 11 and 13, interference with a tooth surface is minimized.

To counteract the taste of the antiviral and/or antibacterial agent(s) when engaged with the dental structure, a miniature container for slowly releasing hygiene-useful material or a medicinal material into the oral cavity may be attached to clip 10.

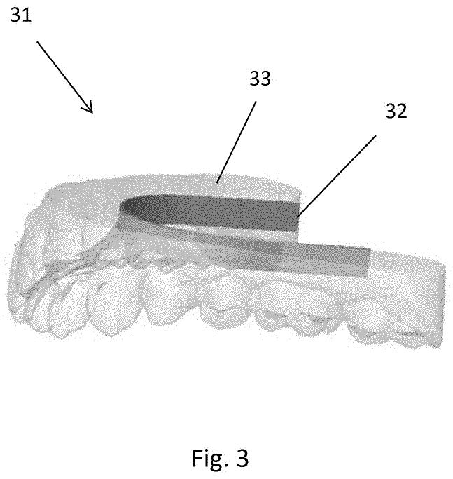

In the embodiment of FIG. 3, the intraoral device is constituted as an insert 32 to a set of dentures 31. Insert 32 is fitted within the denture base 33, which is adapted to engage an edentulous ridge. For example, insert 32 can be made of copper.

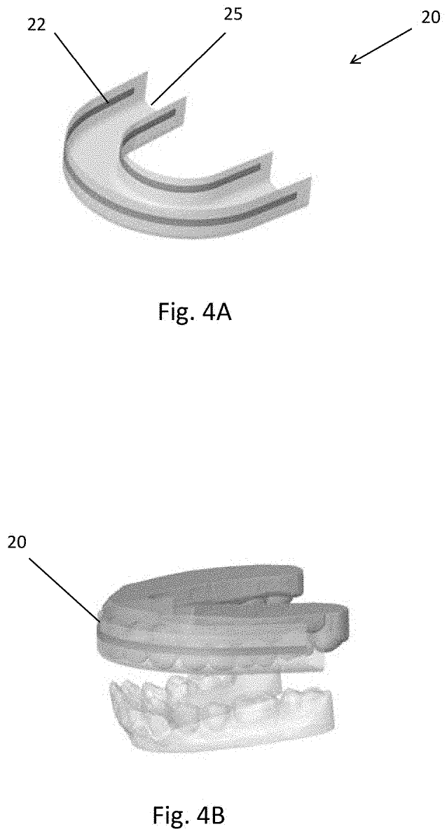

In the embodiment of FIGS. 4A and 4B, the intraoral device is constituted as a mouth guard 20 with an antiviral and/or antibacterial agent(s) in form of strips, e.g., with copper and silver strips as indicated by numerals 22 and 25, respectively.

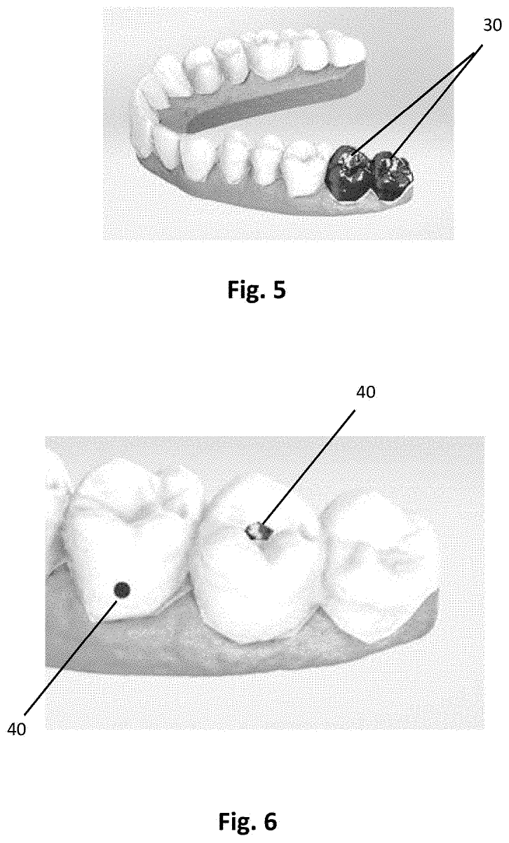

FIGS. 5 and 6 illustrate the intraoral device, according to embodiments of the present invention, in which the antiviral and/or antibacterial agent(s) can be applied into or onto at least one tooth. For example, FIG. 5 illustrates the intraoral device provided with an antiviral and/or antibacterial agent material(s) in the form of metal foils 30 covering the shape of a tooth. In other embodiments, the intraoral device is constituted as metallic foil covering for back teeth (not shown). FIG. 6 illustrates the intraoral device as a restorative and/or filling material 40 which is capable of being applied to a tooth structure from which a carious lesion has been removed.

According to an embodiment of the invention, the intraoral device is constituted as a sealant which comprises the antiviral and/or antibacterial and/or antifungal agent material(s). For example, the sealant can be in form of a thin, plastic coating painted on the surfaces of teeth with suspended nanoparticles made of the antiviral and/or antibacterial and/or antifungal agent material(s). According to some embodiments of the invention, the coating may have a color, thus it would make most sense to apply the coating to less visible surfaces of the teeth, for example, on the back of the molars or the back of the incisors. The sealant may bond into the depressions and grooves of the teeth, forming a protective shield over the enamel of each tooth.

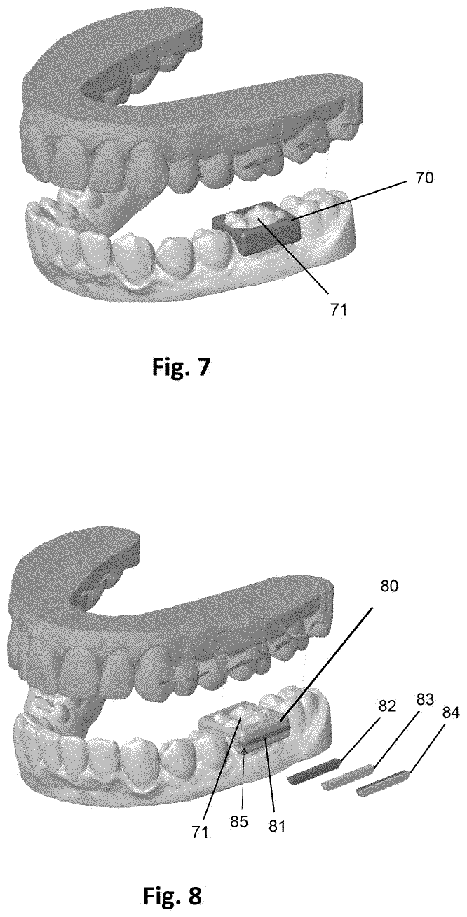

FIG. 7 schematically illustrates the intraoral device 70 made of antiviral and/or antibacterial ion releasing material and placed around a tooth 71, according to an embodiment of the invention. Intraoral device 70 is an example of a metal device that is molded around tooth 71 yet removable. Device 70 can be made of one material, or more than one material (e.g., a combination of copper and silver).

FIG. 8 schematically illustrates an intraoral device 80 comprising interchangeable antiviral and/or antibacterial ion releasing cartridges that is placed around tooth 71, according to embodiments of the present invention. Device 80 is adapted to receive a cartridge that comprises ion releasing material(s) (e.g., as indicated by numerals 81-84). In this embodiment, device 80 comprises a groove 85 into which an antiviral ions releasing cartridge 81 is installed where cartridge 81 is removable (e.g., by sliding throughout groove 85) and can be replaced with other antiviral ions releasing cartridge (e.g., by a cartridge 82, 83 or 84). Of course, each of cartridges 81, 82, 83 and 84 may comprise different types of ion releasing metals, in accordance with a desirable effect thereof. For example, cartridge 82 may comprise copper, cartridge 83 may comprise silver and cartridge 84 may comprise a combination of two or more materials (e.g., copper and silver). Whereas future researches may find beneficial effects correlated to the presence of certain ions, corresponding ion releasing metals can be readily embedded into cartridges 81-84 and be used within device 80.

According to an embodiment of the invention, device 80 could also be semi-permanent (i.e., by semi-permanent it means that device 80 can be adhered to the tooth but removed with some force, e.g., by a dental professional).

According to an embodiment of the invention, devices 70 and 80 are adapted to be removeably installed within an oral cavity of a user. For example, device 80 can be made of polymer material and shaped to be molded around at least one tooth (e.g., detachably attached to tooth 71 or other suitable dental structure within the oral cavity of the user by a dedicated adhesive material suitable to be used within the oral cavity of a human). In this example, device 80 comprises a polymer base that is adhered to the tooth, and the metal cartridges 81-84 are easily replaced.

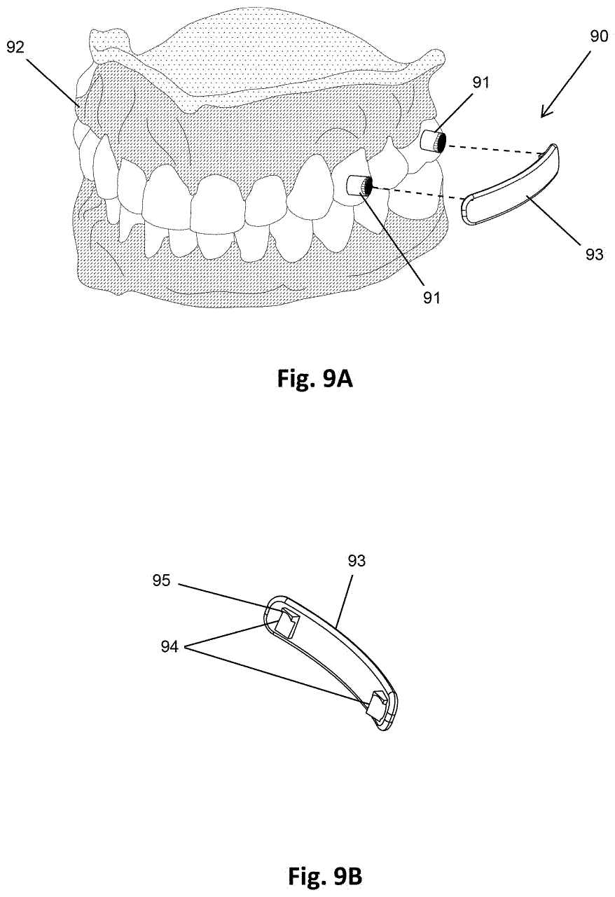

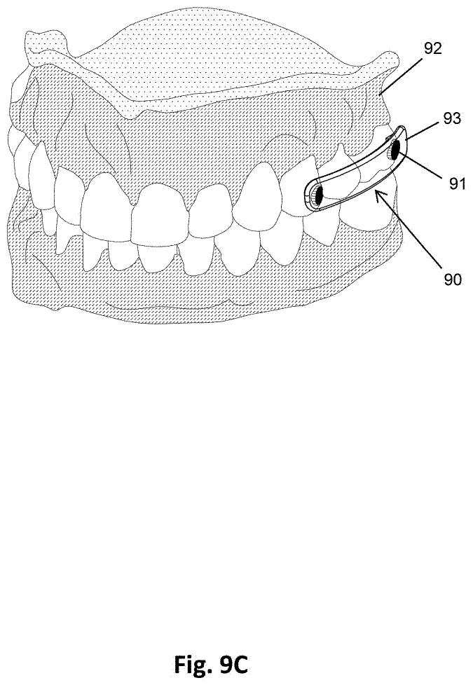

FIGS. 9A-9B schematically illustrate an intraoral device 90, according to an embodiment of the invention. Device 90 comprises an interchangeable antiviral and/or antibacterial ion-releasing plate 93 and one or more bases 91 adapted to be attached to the user's teeth, e.g., by mechanical means. For example, base 91 may be adhered to the user's teeth by dental/biological glue.

Plate 93 comprises one or more attachment elements 94 corresponding to bases 91 and adapted to be magnetically attached to bases 91, and thereby, plate 93 can be removeably attached to a user's teeth or soft tissue lining of the mouth (an illustration of the anatomy of the oral cavity that shows the teeth and gums is indicated by numeral 92). Furthermore, elements 94 may comprise stabilizing protrusions 95 shaped correspondingly to bases 91, for providing an increased firm attachment thereto.

Plate 93 may comprise one or more ion-releasing metals, where different interchangeable plates 93 may be used in accordance with a desirable effect thereof. Plate 93 is curved in accordance with the user's oral cavity structure. Of course, plate 93 may be fabricated with a generic curvature, or be designed with a plurality of predetermined oral cavity curvatures from which users may select the most suitable plate 93 (i.e., which best matches their individual oral cavity structure) or specifically fabricated to fit individual users.

FIG. 9C shows intraoral device 90 in an assembled state, wherein plate 93 is attached to the user's oral cavity 92 by bases 91. It is noted that the external surface of plate 93 (i.e., with respect to the user's oral cavity) is aimed outwardly. Thus, when the user's mouth is opened, ions are released by plate 93 towards incoming air and engage potentially carried viruses thereon.

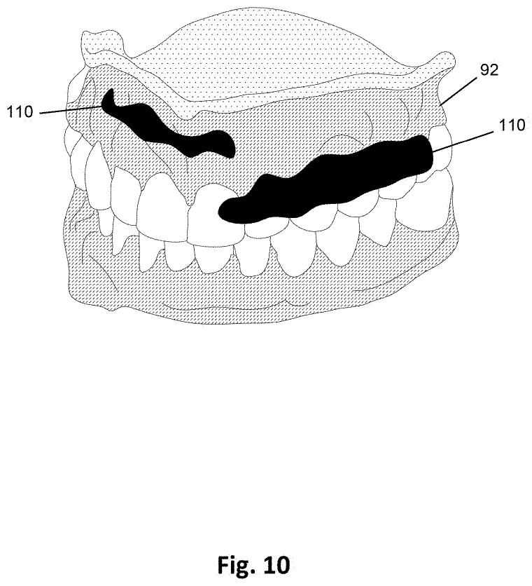

FIG. 10 schematically illustrates an intraoral device 110 provided in a malleable gum-like form, according to an embodiment of the invention. Device 110 is detachably adhered to the user's teeth and/or soft tissue lining of the mouth, by being pressed thereto for adapting to the teeth and gum shape. Device 110 is made from a partially adhesive material that comprises ion-releasing material.

Device 110 is particularly advantageous in cases where a person suffers from gum disease, whereas device 110 may be attached to a wounded/exposed area (e.g., post-gum surgery), and ions released by device 110 may provide additional protection.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. An intraoral device, comprising an antiviral and/or antibacterial and/or antifungal ion releasing agent material(s) which, when said device is exposed to the oral cavity and to any gases, liquids and solids passing through the oral cavity, and is capable of inactivating viruses and/or bacteria and/or fungi penetrating into the oral cavity.

2. The device according to claim 1, wherein the antiviral and/or antibacterial ion releasing agent materials(s) include one or more of the following materials: solid-state copper (Cu), silver (Ag), copper(I) oxide (Cu₂O), copper(II) oxide (CuO), silver nitrate (AgNO₃), silver oxide (Ag₂O), copper(II) chloride (CuCl₂), copper(I) chloride (CuCl), Zinc-Oxide, silver nitrate (AgNO₃) and silver sulfide (Ag₂S) in combinations with natural/synthetic polymers and/or ceramics, or any combination thereof.

3. The device according to claim 1, which is a dental clip that comprises the antiviral and/or antibacterial agent material(s) that are removable and engages a dental structure.

4. The device according to claim 3, wherein the dental clip is configured as a single continuous loop whose peripheral element completely encircles the dental structure.

5. The device according to claim 4, wherein the peripheral element completely encircles a unit of one or more teeth.

6. The device according to claim 4, wherein the peripheral element completely encircles a unit of one or more teeth of a set of dentures or of a dental bridge.

7. The device according to claim 4, wherein the peripheral element comprises two opposed interproximal elements, two opposed buccolingual elements, and a curved portion continuously extending between corresponding interproximal and buccolingual elements.

8. The device according to claim 1, which is an insert made with the antiviral and/or antibacterial agent material(s) and embedded in a base of a set of dentures that extends throughout the dental arch.

9. The device according to claim 8, wherein the insert extends interproximally.

10. The device according to claim 9, wherein the insert extends interproximally throughout the dental arch.

11. The device according to claim 1, wherein the antiviral and/antibacterial agent material(s) are in the form of and covering the shape of the tooth.

12. The device according to claim 1, wherein the antiviral and/antibacterial agent material(s) are restorative material(s) capable of being applied to a tooth structure from which a carious lesion has been removed.

13. The device according to claim 1, wherein the device is adapted to be installed on full or partial dentures or any other pre-existing intraoral device, or on at least one artificial or non-artificial tooth.

14. The device according to claim 1, further comprising at least one cartridge that comprises ion releasing material.

15. The device according to claim 14, wherein the at least one cartridge is interchangeable.

16. The device according to claim 14, wherein the at least one cartridge comprises combination of two or more different types of ion releasing materials.

17. The device according to claim 1, wherein the device is adapted to receive at least one interchangeable cartridge that comprises ion releasing material.

18. The device according to claim 1, wherein the intraoral device is an interchangeable ion-releasing magnetically attached plate.

19. The device according to claim 1, wherein the intraoral device is provided in a malleable gum-like form, detachably adhered by pressing onto a user's oral cavity.
